(19) United States
(12) Patent Application Publication
(10) Pub. No.: US 2021/0128207 A1
(43) Pub. Date: May 6, 2021

(54) BONE COMPRESSION PLATE SYSTEM AND METHOD OF USE

(71) Applicants: Jorge L. Orbay, Miami, FL (US); Alexander N. Gil, Miami, FL (US); Brian A. Smith, Miami, FL (US)

(72) Inventors: Jorge L. Orbay, Miami, FL (US); Alexander N. Gil, Miami, FL (US); Brian A. Smith, Miami, FL (US)

(21) Appl. No.: 16/751,378

(22) Filed: Jan. 24, 2020

Related U.S. Application Data
(60) Provisional application No. 62/930,974, filed on Nov. 5, 2019.

(51) Int. Cl. A61B 17/80

(52) U.S. Cl. CPC A61B 17/8004; A61B 17/809

(57) ABSTRACT

Disclosed is a bone compression plate system comprising a substantially elongated first plate having a bone attachment portion and a anchoring portion, the bone attachment portion comprising one or more holes adapted to receive screws for attachment to a bone, the anchoring portion comprising a compression anchor; a substantially elongated second plate having a bone attachment portion and a anchoring portion, the bone attachment portion comprising one or more holes adapted to receive screws for attachment to a bone, the anchoring portion comprising a compression anchor; and a compression screw comprising a male component and a female component, each of the male and female components comprising a head, a shaft, and a tip, the tip of the male component adapted for threaded engagement with the tip of the female component, the head of the male component adapted to engage into, and be retained by the compression anchor in the first plate, and the head of the component adapted to engage into, and be retained by the compression anchor in the second plate.

### CLAIM OF PRIORITY

This application is being filed as a non-provisional patent application under 35 U.S.C. § 111(a) and 37 CFR § 1.53(b). This application claims priority under 35 U.S.C. § 119(e) to U.S. provisional patent application Ser. No. 62/930,974 filed on Nov. 5, 2019, the contents of which are incorporated herein by reference.

### FIELD OF INVENTION

The invention relates generally to bone fusion devices and systems, and, in particular, to a plate and screw system adapted to stabilize and compress a fractured bone epiphysis, including but not limited to, the humeral distal epiphysis (trochlea).

### BACKGROUND OF THE INVENTION

In some types of bone fractures, for example fractures involving multiple fragments in the epiphysis, it is desirable to stabilize the fracture using compression of the bone fragments after provisional reduction. Described herein is a system comprising two plates to be applied bilaterally to the bone in question, the two plates adapted for coupling to a two-piece compression screw that traverses the bone fragments and whose ends are held captive by the plates.

It is one object of the instant invention to provide a bone compression system and methods for use thereof that is adapted to stabilize a multiple fragment fracture in the bone.

### SUMMARY OF THE INVENTION

Disclosed is a bone compression plate system comprising a substantially elongated first plate having a bone attachment portion and a anchoring portion, the bone attachment portion comprising one or more holes adapted to receive screws for attachment to a bone, the anchoring portion comprising a compression anchor; a substantially elongated second plate having a bone attachment portion and a anchoring portion, the bone attachment portion comprising one or more holes adapted to receive screws for attachment to a bone, the anchoring portion comprising a compression anchor; and a compression screw comprising a male component and a female component, each of the male and female components comprising a head, a shaft and a tip, the tip of the male component adapted for threaded engagement with the tip of the female component, the head of the male component adapted to engage into, and be retained by the compression anchor in the first plate, and the head of the component adapted to engage into, and be retained by the compression anchor in the second plate.

Also disclosed is a method for reducing a bone fracture having two or more bone fragments comprising the steps of: attaching using bone screws a first plate to a first side of the bone, the first plate comprising a substantially elongated body having a bone attachment portion and a anchoring portion, the bone attachment portion comprising one or more holes adapted to receive the bone screws, the anchoring portion comprising a compression anchor; attaching using bone screws a second plate to a second side of the bone, the second plate comprising a substantially elongated body having a bone attachment portion and a anchoring portion, the bone attachment portion comprising one or more holes adapted to receive the bone screws, the anchoring portion comprising a compression anchor; assembling the bone fragments and aligning the assembled bone fragments between the compression anchor of the first plate and the compression anchor of the second plate; drilling a bore through said assembled bone fragments, the bore extending from the compression anchor of the first plate to the compression anchor of the second plate; inserting a male component of a compression screw through the compression anchor of the first plate and into the bore, the male component of the compression screw comprising a head, a shaft, and an externally threaded tip, the head of the male component adapted to engage into, and be retained by the compression anchor in the first plate; inserting a female component of a compression screw through the compression anchor of the second plate and into the bore, the male component of the compression screw comprising a head, a shaft, and an internally threaded opening, the head of the female component adapted to engage into, and be retained by the compression anchor in the second plate; and progressively engaging the externally threaded tip of the male component of the compression screw with the internally threaded opening of the female component of the compression screw thereby compressing the assembled bone fragments.

### BRIEF DESCRIPTION OF THE DRAWINGS

FIG. 1 is an assembled view of an embodiment of the present system adapted for stabilization of a fracture in the distal humeral epiphysis with the fracture fragments partially transparent to permit visualization of the two-piece compression screw.

FIG. 2 is the embodiment shown in FIG. 1 with the bones completely removed to provide full visualization of the two-piece compression screw.

FIG. 3 is a first plate in accordance with the embodiment of the present invention shown in FIG. 1.

FIG. 4 is a second plate in accordance with the embodiment of the present invention shown in FIG. 1.

FIG. 5 is a disassembled two-piece compression screw in accordance with embodiments of the present invention.

FIG. 6 shows a rear view of the embodiment shown in FIG. 2

FIG. 7 shows a top view of the embodiment shown in FIG. 2.

FIGS. 8A-8B show a locking cap in accordance with one embodiment of the present invention.

### DETAILED DESCRIPTION OF THE INVENTION

The system comprises a first bone plate that is adapted to be affixed to one side of the bone, a second plate that is adapted to be affixed to an opposite side of the bone, and a two-piece screw that bridges the two plates and provides compressional stability to the bone fragments. The two-piece compression screw is constrained by anchors provided in each of the two plates and optionally retained by locking caps installed after insertion.

The totality of either of the two plates does not necessarily need to be affixed to a single side of the bone. The plates may be adapted for attachment to the front or back of the bone but still provide for the compression screw anchors to be provided on the opposing aspects of the fractured epiphysis.

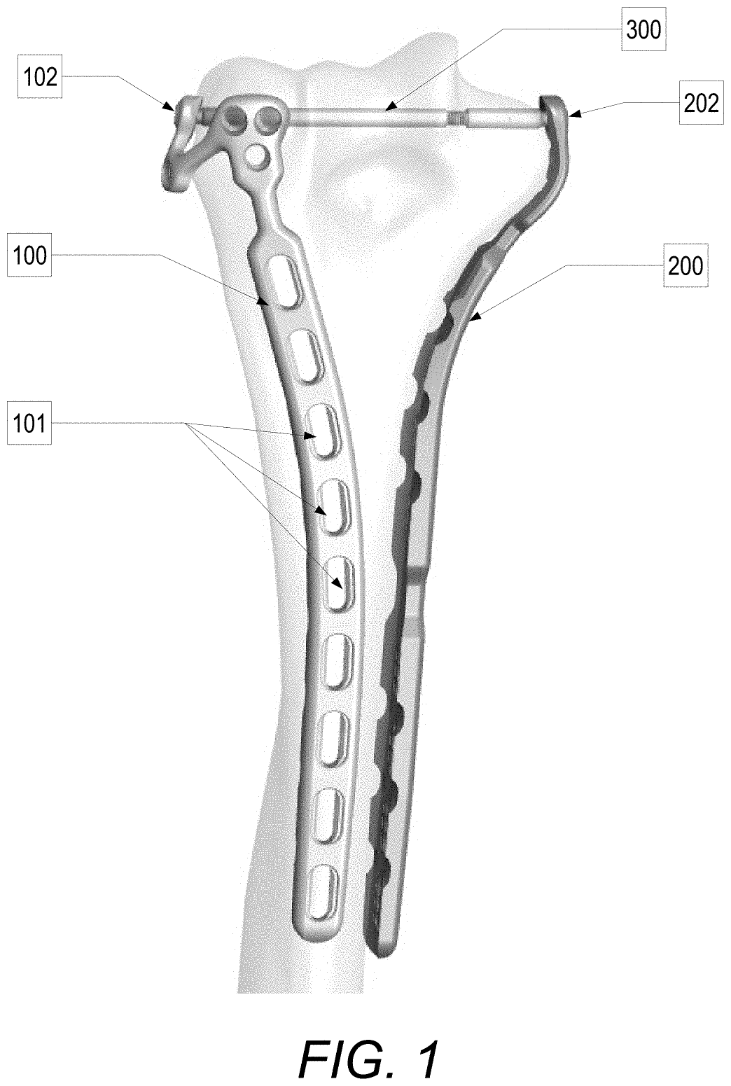

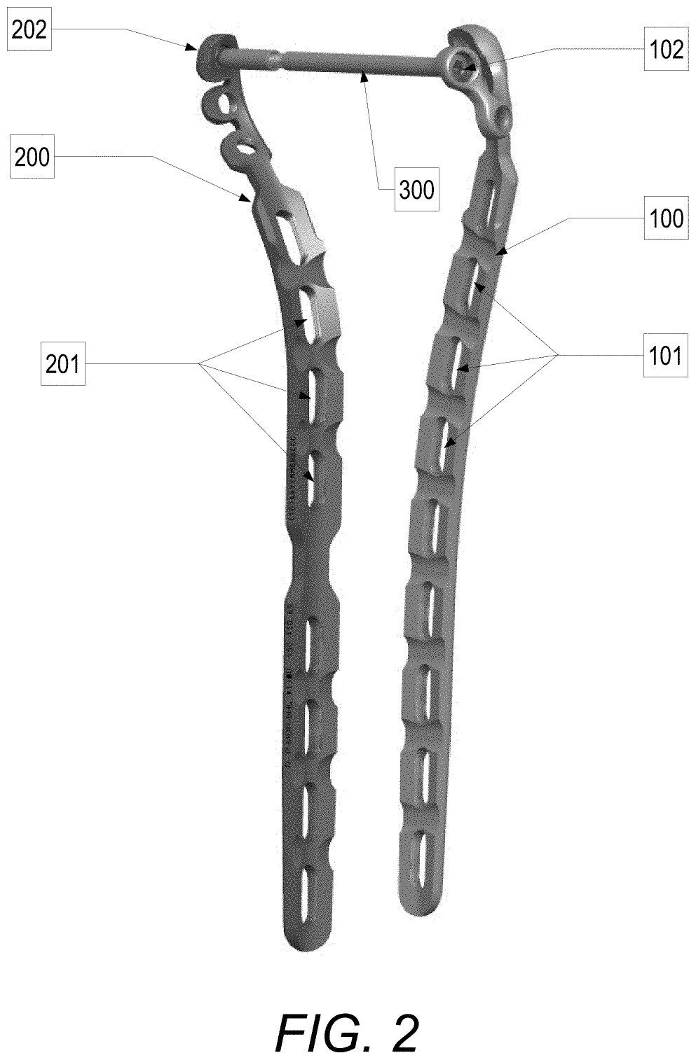

Referring to FIGS. 1 and 2, shown is an embodiment of the present system adapted for stabilization of a fracture in the distal humeral epiphysis, or trochlea. FIG. 1 illustrates the system installed on the bone with the fracture fragments partially transparent to permit visualization of the two piece compression screw. FIG. 2 shows the same embodiment from a different angle and with the bone completely removed for better visualization.

As is shown in FIGS. 1 and 2 the system comprises two bone plates (100, 200) each providing bone attachment holes (101,201) and a compression screw anchor (102,202) at or near the terminal end of the plate. It should be noted that compression screw anchors (102,202) need not be located near an end of the plate and may optionally be located in the middle of the plate. The system further comprises a two-piece compression screw (300) which bridges the compression screw anchors (102,202) across the fragments to be stabilized.

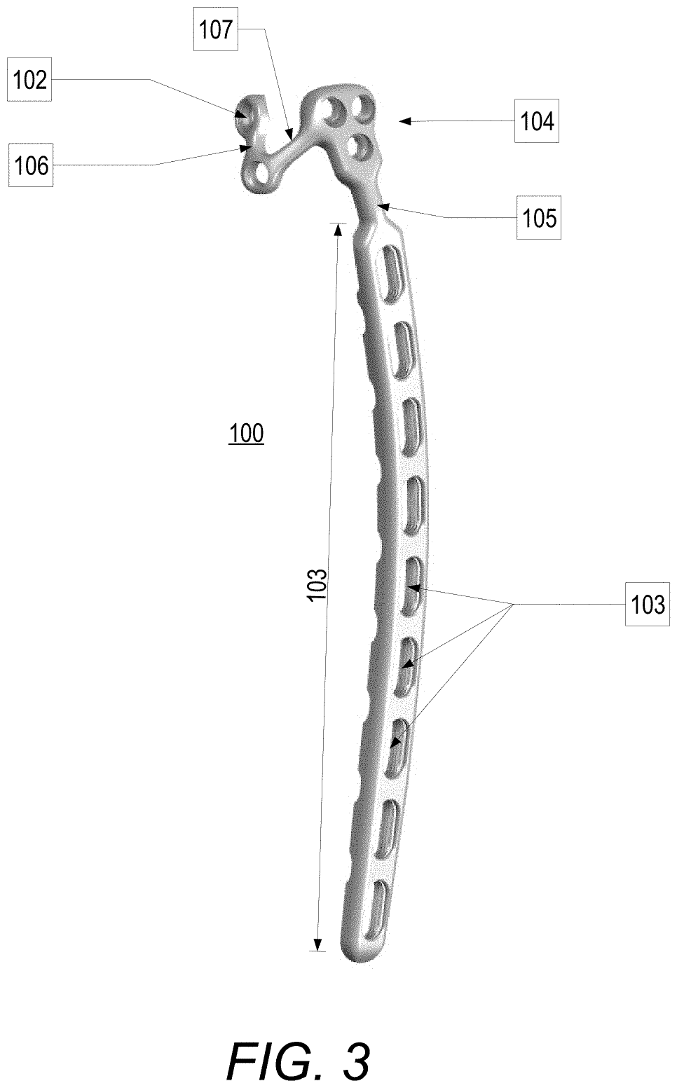

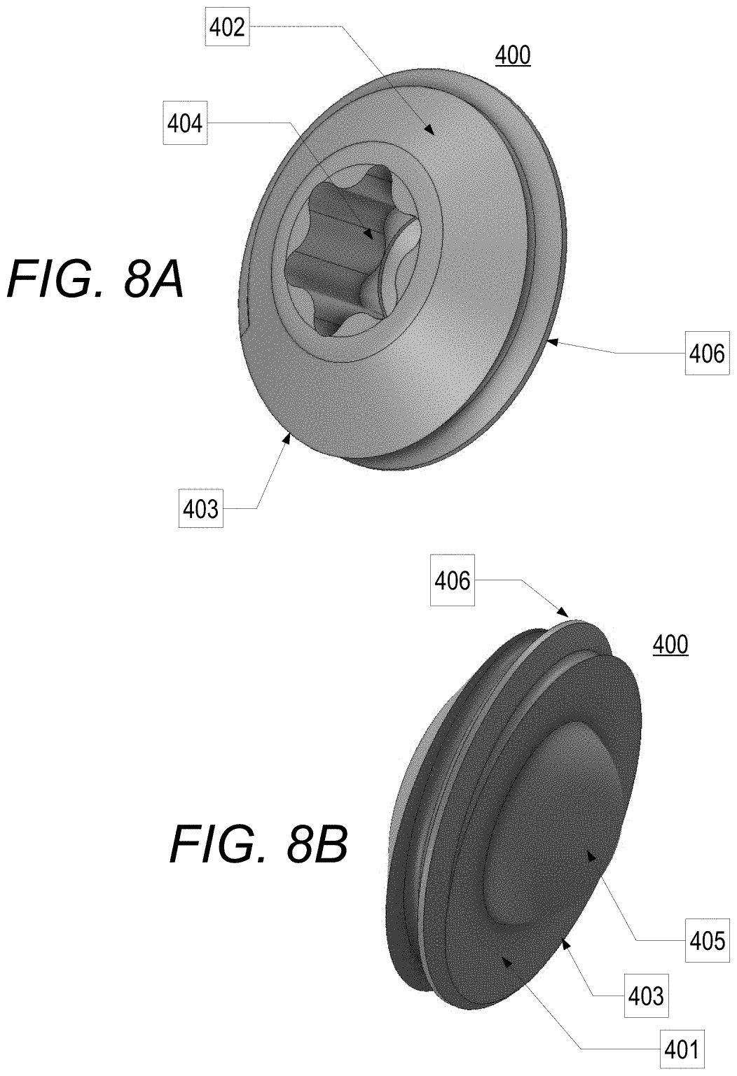

Shown in FIG. 3 is first plate (100) which comprises a bone attachment portion (103) which includes threaded or unthreaded bone holes or slots (101)(or a combination of threaded and unthreaded holes or slots) for affixing the plate to the bone. First plate (100) further comprises compression anchor (102) that is located at the anchoring portion (104) of the first plate. It should be noted that compression screw anchor (102) need not be located near an end of the plate and may optionally be located in the middle of the plate. Compression anchor (102) may optionally include internal threads for attachment of a locking cap (400) shown in greater detail in FIG. 8. The bone attachment portion (103) and the anchoring portion (104) of the plate may be optionally connected through a neck (105) which facilitates bending of the plate to conform to the bone. Additional necks (not shown) may also optionally be provided in the bone attachment portion (104) to further increase flexibility of the bone attachment portion. The anchoring portion (104) may also include one or more necks (106,107) to permit the anchoring portion to conform to the bone and to allow for optimal placement of the compression anchor (102).

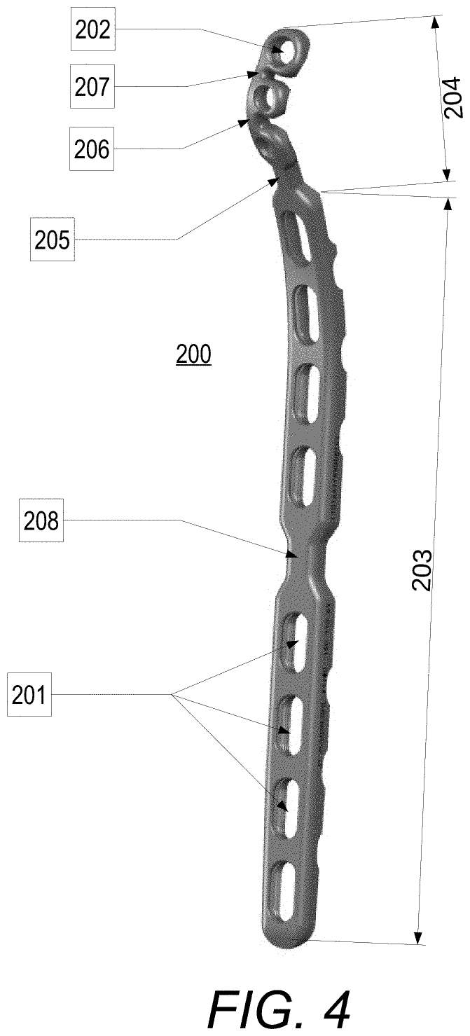

Shown in FIG. 4 is second plate (200) which comprises a bone attachment portion (203) which includes threaded or unthreaded bone holes or slots (201)(or a combination of threaded and unthreaded holes or slots) for affixing the plate to the bone. Second plate (200) further comprises compression anchor (202) located at the anchoring portion (204) of the second plate. It should be noted that compression screw anchor (202) need not be located near an end of the plate and may optionally be located in the middle of the plate. Compression anchor (202) may optionally include internal threads for attachment of a locking cap (400) shown in greater detail in FIG. 8. The bone attachment portion (203) and the anchoring portion (204) of the plate may be optionally connected through a neck (205) which facilitates bending of the plate to conform to the bone. Additional necks (208) may also optionally be provided in the bone attachment portion (204) to further increase flexibility of the bone attachment portion. The anchoring portion (204) may also include one or more necks (206,207) to permit the anchoring portion to conform to the bone and to allow for optimal placement of the compression anchor (202).

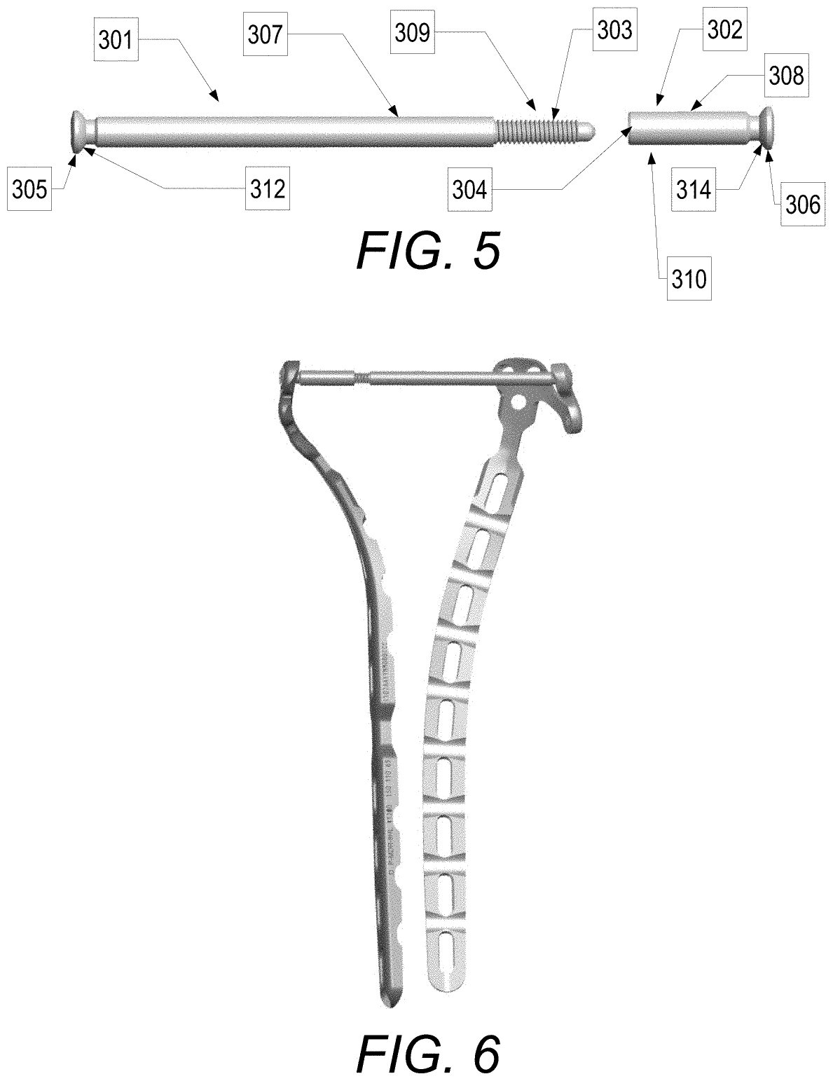

Shown in FIG. 5 is a disassembled view of two-part compression screw (300). It comprises a male (301) and female (302) components which are adapted for engagement. The male and female components (301,302) each respectively comprise a head (305,306), a shaft (307,308), and a tip (309,310) The tip (309) of the male component (301) comprises a threaded end (303) which is adapted to engage a threaded hole (304) in the tip (310) of the female component (302). Heads (305,306) are adapted to engage into and be constrained by compression anchors (102, 202) on the first and second plates. Both heads (305,306) may comprise a driving feature adapted to be engaged by a screwdriver for fastening. Alternatively, one of the heads (305,306) may comprise a driving feature while the other comprises a rotation restricting feature that, in cooperation with its corresponding compression anchor (102 or 202) restricts rotational movement of the head to ease one-handed fastening. The rotation restricting feature may comprise a key in the compression anchor that engages a corresponding key-hole in the head, or vice-versa. The rotation restricting feature may also comprise a shaped recess (such as a polygon, multi-lobed, star, and the like) in the compression anchor which matches a corresponding shape in the head.

One or both of the heads (305,306) of the male (301) and female (302) compression screw (300) components may optionally include interior abutting surfaces (312,314) having generally concave (not shown) or convex (shown) shapes. The abutting surfaces would match a corresponding shape on a bearing surface (not shown) of the compression anchors (102,202) permitting the male (301) and/or female (302) components of compression screw (300) to change orientation, or swivel, with respect to the first and/or second plates (100,200) as necessary or desirable to reach optimal alignment of the male (301) and female (302) compression screw (300) components upon installation.

It will be observed that as the male and female components (301,302) are progressively engaged, the total length of the compression screw (300) is progressively reduced. This in turn provides compression when engaged with compression anchors. Once compression screw (300) is in place, it may be optionally retained by installing locking caps (400, FIG. 8) at compression anchors (102,202).

Shown in FIG. 8 is a locking cap (400) in accordance with some embodiments of the present invention. Locking cap (400) comprises a disk shaped body having an internal surface (401), an external surface (402) and a periphery (403). External surface comprises a driving feature (404) adapted to be engaged by a screwdriver or other driving tool (not shown) for fastening. Internal surface comprises a protrusion (405) adapted to abut the head (305,306) of compression screw (300) once the locking cap (400) is fully installed on compression anchors (102, 202). Periphery (403) comprises an external screw thread (406) adapted to engage internal threads on compression anchors (102, 202).

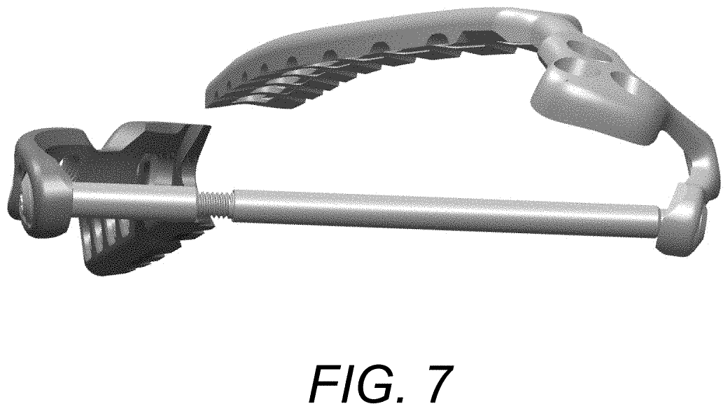

Additional details of the present invention are provided in FIGS. 6 and 7 which illustrate the following: FIG. 6 shows a rear view of the embodiment shown in FIG. 2; FIG. 7 shows a top view of the embodiment shown in FIG. 2.

Method of Use

In operation, the first plate (100) and the second Plate (200) are placed on the diaphysis of the bone to be treated so as to generally align compression anchors (102,202) opposite each other on either side of the fractured epiphysis. Plates (100,200) are fastened to the diaphysis using bone screws (not shown) through bone holes or slots (109,209).

The fractured bone fragments are then reduced and assembled, and plates (100,200) are bent so as to align compression anchors (102,202) on either side of the fractured epiphysis. A hole is then made (using a drill or a K-Wire) extending from one of the compression anchors (102,202) to the other, and through the fractured fragments.

The hole is drilled and reamed to an appropriate size to permit shafts (307,308) to be inserted therethrough. The male and female components (301,302) of compression screw (300) are then each inserted through a respective compression anchor (102,202) until they mate inside the hole. The male and female components (301,302) are then screwed together until the desired level of compression is achieved.

Finally, locking caps (400) are inserted and fastened into compression anchors (102,202) over screw heads (305,306) to retain compression screw (300) in place.

Although described above in connection with certain bone types and parts, namely the distal humeral epiphysis, these descriptions are not intended to be limiting as various modifications may be made therein without departing from the spirit of the invention and within the scope and range of equivalent of the described embodiments. Encompassed embodiments of the present invention can be used in all applications where bone fractures can be stabilized through compression of bone fragments.

We claim:

1. A bone compression plate system comprising:
a substantially elongated first plate having a bone attachment portion and a anchoring portion, the bone attachment portion comprising one or more holes adapted to receive screws for attachment to a bone, the anchoring portion comprising a compression anchor;
a substantially elongated second plate having a bone attachment portion and a anchoring portion, the bone attachment portion comprising one or more holes adapted to receive screws for attachment to a bone, the anchoring portion comprising a compression anchor; and
a compression screw comprising a male component and a female component, each of the male and female components comprising a head, a shaft, and a tip, the tip of the male component adapted for threaded engagement with the tip of the female component, the head of the male component adapted to engage into, and be retained by the compression anchor in the first plate, and the head of the component adapted to engage into, and be retained by the compression anchor in the second plate.

2. The bone compression plate system of claim 1 wherein at least one of the one or more holes in either the first plate or the second plate is threaded.

3. The bone compression plate system of claim 1 wherein at least one of the one or more holes in either the first plate or the second plate is unthreaded.

4. The bone compression plate system of claim 1 wherein at least one of the one or more holes in either the first plate or the second plate is an elongated slot.

5. The bone compression plate system of claim 1 wherein at least one of the compression screw's male component head and the compression screw's female component head comprises a driving feature adapted for engagement by a driving tool.

6. The bone compression plate system of claim 1 wherein either the compression screw's male component head or the compression screw's female component head is adapted to engage a rotation restriction feature in either the first portion's compression anchor or the second portion's compression anchor that restricts rotational movement of the compression screw's male component head or compression screw's male component head.

7. The bone compression plate system of claim 6 wherein the rotation restriction feature comprises a key, a key-hole, or a shaped recess.

8. The bone compression plate system of claim 1 further comprising at least one locking cap adapted for threaded engagement with the compression anchor of the first plate or the second plate.

9. The bone compression plate system of claim 1 wherein at least one of the heads of the male component or the female component of the compression screw further comprises an abutting surface having a convex or concave shape, and the compression anchor on one of the first plate and second plate further comprises a bearing surface having a convex or concave shape corresponding to the abutting surface.

10. A method for reducing a bone fracture having two or more bone fragments comprising the steps of:
attaching using bone screws a first plate to a first side of the bone, the first plate comprising a substantially elongated body having a bone attachment portion and a anchoring portion, the bone attachment portion comprising one or more holes adapted to receive the bone screws, the anchoring portion comprising a compression anchor;
attaching using bone screws a second plate to a second side of the bone, the second plate comprising a substantially elongated body having a bone attachment portion and a anchoring portion, the bone attachment portion comprising one or more holes adapted to receive the bone screws, the anchoring portion comprising a compression anchor;
assembling the bone fragments and aligning the assembled bone fragments between the compression anchor of the first plate and the compression anchor of the second plate;
drilling a bore through said assembled bone fragments, the bore extending from the compression anchor of the first plate to the compression anchor of the second plate;
inserting a male component of a compression screw through the compression anchor of the first plate and into the bore, the male component of the compression screw comprising a head, a shaft, and an externally threaded tip, the head of the male component adapted to engage into, and be retained by the compression anchor in the first plate;
inserting a female component of a compression screw through the compression anchor of the second plate and into the bore, the male component of the compression screw comprising a head, a shaft, and an internally threaded opening, the head of the female component adapted to engage into, and be retained by the compression anchor in the second plate; and
progressively engaging the externally threaded tip of the male component of the compression screw with the internally threaded opening of the female component of the compression screw thereby compressing the assembled bone fragments.
